(19) United States
(12) Patent Application Publication
(10) Pub. No.: US 2026/0272375 A1
(43) Pub. Date: Sep. 17, 2026

(54) DIAGNOSTIC SYSTEM FOR DETERMINING A CLINICALLY RELEVANT SEVERITY OF A SLEEP-RELATED BREATHING DISTURBANCE FROM A PATIENT SIGNAL BY WEIGHTING RESPIRATORY EVENTS WITH RESPIRATORY EVENTS THAT ARE CLOSE IN TIME

(71) Applicant: DIAMETOS GMBH, Potsdam (DE)

(72) Inventor: Christoph JANOTT, Berlin (DE)

(21) Appl. No.: 19/472,721

(22) PCT Filed: Mar. 28, 2024
(86) PCT No.: PCT/EP2024/058703

§ 371 Date: Oct. 6, 2025

(30) Foreign Application Priority Data
Apr. 6, 2023 (DE) 10 2023 108 915.3

(51) Int. Cl. A61B 5/00; A61B 5/05; A61B 5/08

(52) U.S. Cl. CPC A61B 5/4818; A61B 5/05; A61B 5/08; A61B 5/7264; A61B 5/7278

(57) ABSTRACT

The present invention relates to a diagnostic system for determining a clinically relevant severity of a sleep-related breathing disturbance from a patient signal of a sleeping patient, comprising: a sensor unit which captures a patient parameter and generates a patient signal; a first detector unit which detects respiratory events in the patient signal and generates a corresponding respiratory event signal therefrom; and a second detector unit which comprises the following: a weighting determination unit which detects, in the respiratory event signal at a particular respiratory event within a time window in which the respiratory event takes place, respiratory events that are close in time and, depending on a number and/or energy of the particular respiratory events that are close in time, generates a weighting signal; a weighting unit which weights the respiratory event signal with a weighting signal; and a severity determining unit which determines the clinically relevant severity from an integral via the weighted respiratory event signal.

According to the preamble of Claim 1, the present invention relates to a method for determining a clinically relevant severity of a sleep-related breathing disturbance from a patient signal.

The clinically relevant severity of the sleep-related breathing disturbance is determined, in the state of the art, from a number of apnoeas and hypopnoeas, divided by a measurement time in hours. Apnoeas are defined as flows of breathing reduced by at least 90% with respect to an initial value over at least 10 seconds. Hypopnoeas are defined as flows of breathing reduced by at least 30% with respect to the initial value over at least 10 seconds in combination with oxygen saturation reduced by at least 3%. Other definitions require an oxygen saturation reduced by at least 4% or simultaneous occurrence of an arousal. In the following, apnoeas and hypopnoeas will be summarized as respiratory events. The measurement time is preferably the duration from the beginning to the end of measurement, without the observation of sleep-wake cycles. Alternatively preferred, the measurement time is a sleeping time of the patient.

Commonly, the clinically relevant severity of the sleep-related breathing disturbance is determined in the state of the art as follows:
a) slight sleep apnoea with 5 -15 apnoeas or hypopnoeas per hour of measurement time;
b) medium-degree sleep apnea with 15-30 apnoeas or hypopnoeas per hour of measurement time;
c) severe sleep apnoea with more than 30 apnoeas or hypopnoeas per hour of measurement time.

The clinically relevant severity can be determined in portions over the measurement time or over the entire time of measurement. Alternatively or in addition, the severity can also comprise a plurality of severity values which can be determined, for instance, over different periods of the time of measurement.

For determining breathing and a breathing disturbance of the patient, a patient signal will be recorded by a sensor unit, which signal represents at least one physiological parameter of the patient. Preferably, the patient signal is a microphone signal representing the breathing of the patient. The patient signal can also comprise other and/or additional measurement signals, such as an SpO2 signal of a pulse oximeter, a transmitter belt signal and/or a motion sensor signal.

Normally, an intensity signal is formed from the patient signal which is, for instance, an RMS output of the patient signal. The intensity signal is then fed into a threshold value detector which detects the respiratory events if the intensity signal falls below a predetermined threshold value. The threshold value can be determined, for instance, by detecting a periodic amplitude of the intensity signal during a non-disturbed sleeping time and from there determining which is the minimum amplitude which the intensity signal does normally not fall below; then the predetermined threshold value can be equated to the minimum amplitude multiplied by a tolerance factor less than 1.

AT 520 925 A4 discloses a method of detecting breathing interruptions of the patient, where during the patient's sleep a breathing movement measurement value of the patient's movements and an oxygen saturation value of the patient are determined and correlated. A breathing interruption is detected if the breathing movement measurement value drops and the oxygen saturation value drops in addition.

US 2012_0071 741 A1 discloses a method of determining an apnoea/hypopnoea index in which method both the respective breathing and snoring noises and the respective oxygen saturation value of the patient are taken into account.

US 2024_0 008 765 A1 discloses a method of determining apnoeas, in which method respective ECG signals and their course over time are evaluated und taken into account.

Especially during automatic detection of the respiratory events using only one parameter or only a few physiological parameters, respiratory events are often counted erroneously or not recognized, so that a resultant detection of the severity of the sleep-related breathing disturbance of the patient is erroneous as well. In order to prevent this, the predetermined threshold value for the threshold value detector is, in some embodiments of the state of the art, determined in a complicated manner as a prediction value. Furthermore, influences like surrounding noises can impair determination of the clinical severity from acoustically determined physiological parameters.

The task of the invention, aimed at eliminating drawbacks from the state of the art, is therefore to provide a diagnostic system which detects a clinically relevant severity of a sleep-related breathing disturbance as precisely and reliably as possible by means of a patient signal.

This task is solved by a diagnostic system according to the features of independent Claim 1. Other advantageous embodiments of the invention are indicated in the dependent Claims.

According to the invention, a diagnostic system for detecting a clinically relevant severity of a sleep-related breathing disturbance from a patient signal of a sleeping patient is made available, comprising:
a) a sensor unit which records at least one patient parameter and generates a respective patient signal from it;
b) a first detector unit having a first signal processing which generates an intensity signal from the patient signal, and a second signal processing which detects respiratory events in the intensity signal and generates a corresponding respiratory event signal; and
c) second detector unit adapted to determine the clinically relevant severity from the respiratory event signal;
d) the second detector unit having:
d1) a weighting determination unit adapted to detect the respiratory events in the respiratory event signal and to determine, for each respiratory event, a corresponding weighting signal by determining for the respective respiratory event close respiratory events lying within a corresponding predetermined time window which also comprises the respective respiratory event, and generating the respective weighting signal depending on a number and/or an energy of the respective respiratory events that are close in time, and at least partially rising;
d2) a weighting unit which weights the respiratory event signal with the weighting signal and generates therefrom a weighted respiratory event signal with correspondingly weighted respiratory events; and
d3) a severity detection unit which detects the clinically relevant severity as an integral value over the weighted respiratory event signal.

What is particularly advantageous is that before further evaluation on clinically relevant severity, the respiratory events are weighted in such a way that singular detected respiratory events and/or events low in energy are weighted lower, contributing little to clinically relevant severity, whereas frequently detected respiratory events within the time window and/or events higher in energy are weighted higher and accordingly contribute more to clinically relevant severity. Thus, erroneously detected respiratory events can be well attenuated or suppressed as artifacts, which are normally singular events, whereas a larger number of detected events occurring more frequently contribute to a larger extent to determining the clinically relevant severity of the breathing disturbance. An occurrence of a quick series of artifacts is usually rare.

Therefore, the method according to the invention is robust with regard to artifacts in correctly detecting the respiratory events due to weighting of the respiratory events, with singular erroneous detections accordingly distorting the clinically relevant severity only to a lesser extent.

Preferably, the weighting determination unit is adapted to shift the predefined time window for the respective respiratory event on the time axis and arrange it such that the largest possible number of respiratory events close in time are at least partly located within the time window. Preferably, in this process, the time window of the time of the respective respiratory event is shifted on the time axis to the left and then successively to the right in such a way that the time of the event is still in the time window, with each time determining the number of respiratory events close in time which are located entirely or at least partially within the time window; then the largest number of respiratory events close in time in the respective time window is used for determining the weighting signal at the respective time of the event. Of course, for each arranged time window the respective number of respiratory events close in time is first stored and then the maximum number of all possible time windows comprising the respective respiratory event is used for determining the weighting signal.

Alternatively preferably, the weighting determination unit is adapted to shift the predefined time window for the respective respiratory event on the time axis and arrange it such that the highest possible energy of the respective event and of the respiratory events close in time is present in the time window. Preferably, in this process, the time window of the time of the respective respiratory event is shifted on the time axis to the left and then successively to the right in such a way that the time of the event is still in the time window, with the energy of the respiratory event and of the respiratory events close in time which lie entirely or at least partially within the time window being determined each time; after this, the highest determined energy is used for determining the weighting signal at the respective time of the event.

For clarity, the energy of the respective respiratory event is a product of an amplitude and a duration of the respiratory event signal over the respiratory event. The energy of the respiratory event and of the other respiratory events close in time is understood to be the sum of all energy components of the respiratory events in the respiratory event signal which are located in the respective time window. With binary amplitude charts of the respiratory event signal, where a respiratory event can be present or not, the energy component is determined from the amplitude which is 1 or 0, multiplied by the time in the respective time window. In case of the binary amplitude, the energy represents the duration of time. However, the respiratory event signal can also be provided with a variable amplitude which can assume a plurality of discrete values so as to be able to map different severities of the respective respiratory event. The energy of the respiratory event signal is frequently a good indicator for robustness of detection of an actual respiratory event. In other words, the probability that the detected event is an artifact and therefore an error is the lower the higher the energy of the respective respiratory event signal is.

It is particularly advantageous to take into account the energy of the respiratory event and of the respiratory events close in time in the respective time window for determining the weighting signal, the clinically relevant severity being closely correlated with an ODI (Oxygen Desaturation Index). The clinically relevant severity determined in this manner is therefore a good measure for the severity of oxygen desaturation during sleep which is clinically relevant.

On the other hand, a clinically relevant severity determined using the number of respective respiratory events and of the respiratory events close in time is more closely correlated with an AHI index also called apnoea hypopnoea index.

Alternatively preferably, the weighting determination unit is adapted to shift the predefined time window as a sliding period of time along a time axis of the respiratory event signal, determining the number of all respiratory events at least partially contained therein and mapping the number in the weighting signal at a predefined time within the time window. For purposes of clarity, the predefined time within the time window is a point in time predefined in relation to the respective time window, e.g. at the beginning, in the middle or at the end of the time window. Preferably, the number is indicated as a value in the weighting signal in the middle of the time window. The number is preferably an integral value; alternatively, it can also be a decimal value.

Alternatively preferably, the weighting determination unit is adapted to shift the predefined time window as a sliding period of time along the time axis of the respiratory event signal, forming an integral value over the energies of all respiratory events at least partly contained therein and mapping the integral value in the weighting signal at the predetermined time within the time window.

Preferably, the weighting determination unit is adapted to determine the weighting signal in dependence on the number of respective respiratory events in the respiratory event signal within the predetermined time window such that the weighting signal increases with the number of respiratory events contained in the time window. Preferably, the weighting signal increases within a predefined range f the number of respective respiratory events. In this process, the weighting signal can be formed both as a linear and as a non-linear function of the number of respiratory events. For instance, a lower threshold value of the number can be provided below which the weighting function is set to zero or to a minimum value. In the same way, an upper threshold value of the number can be provided above which the weighting function is limited. Preferably, the weighting signal is generated in dependence on the number of respective respiratory events close in time within a major range of values of the number of respective respiratory events close in time, increasing therewith. The major range of values is preferably a range which covers at least 50% of a total range of values of the number of respiratory events or which covers a frequency range of the numbers which make up at least 50% of all numbers.

Preferably, the weighting determination unit is adapted to determine the weighting signal in dependence on the energy of the respective respiratory event and of the respiratory events close in time within the predetermined time window such that the weighting signal increases with the energy. Preferably, the weighting signal increases within a predefined range of the energy of the respiratory events close in time. In this process, the weighting signal can be formed both as a linear and as a non-linear function of the energy of the respiratory events. For instance, a lower threshold value of the energy can be provided below which the weighting function is set to zero or to a minimum value. In the same way, an upper threshold value of the energy can be provided above which the weighting function is limited. Preferably, the weighting signal is generated in dependence on the energy of the respective respiratory events close in time within a major range of values of the energy of the respective respiratory events close in time, increasing therewith. The major range of values is preferably a range which covers at least 50% of a total range of values of the energy of the respiratory events or which covers a frequency range of the energies which make up at least 50% of all possible energies.

Preferably, the clinically relevant severity is determined by the second detector unit by means of a respective correction or calibration factor. Preferably, the correction or calibration factor is applied to the clinically relevant severity by the severity determining unit. It is also conceivable, however, that the weighting determination unit takes into account the correction factor in the weighting signal. It is also possible that a function of the weighting signal is determined in dependence on the respective number and/or energy in such a way that the clinically relevant severity comes closest to an actual clinically relevant severity determined by skilled personnel. The weighting signal might be, for instance, a function of a first factor multiplied by the respective number plus a second factor multiplied by the respective energy. An optimization of the first and the second factor, for example by means of an LMS (Least Mean Square) method, can be regarded as a correction applied to the weighting signal, approaching the clinical severity to the actual severity.

Preferably, the correction or calibration factor is determined, using studies with reference values for clinically relevant severities, which values have been determined by experts, in such a way that the clinically relevant severity of the diagnostic system statistically comes as close to the respective reference values as possible, and such that in this manner the clinically relevant severity determined by the diagnostic system preferably comes close either to the respective ODI or AHI value.

It is true that the ODI value could be determined, for instance, using an oximeter, which, however, requires substantive effort and apparatuses; the above invention allows determining the ODI value much easier, for instance using a microphone signal as the patient signal. The microphone signal only needs to be recorded, for example, by a mobile phone, and transferred to the diagnostic system, so that the respective clinically relevant severity of the sleep-related breathing disturbance can be determined. The sensor unit can either be part of an integral diagnostic system or a remote part which is connected, e.g. wirelessly, to the residual part of the diagnostic system.

Preferably, the severity determining unit determines the clinically relevant severity as an integral value over the weighted respiratory event signal in combination with a predetermined correction factor.

Preferably, the correction factor is predetermined from a plurality of respective correction factors as a mean value by equating a plurality of integral values taken from training data sets from the weighted respiratory event signals, multiplied by the respective correction factors, to the respective actual clinically relevant severities which have been determined by the skilled personnel. The correction factors can be determined by formation of quotients. The correction factor is preferably determined using data from a clinical study with sleep patients, the actual clinically relevant severity being determined by skilled personnel.

It is again pointed out that what is particularly advantageous in the method according to the invention is the ease of acquisition of the patient signal, for instance in the form of a microphone signal recorded at the home of the patient by a mobile phone and transferred to the diagnostic system. In this manner, the clinically relevant severity of the sleep-related breathing disorder can be determined without the need to visit a sleep laboratory, simply from home, where errors in the patient signal which might lead to an erroneous detection of respiratory events can to a large extent be suppressed by weighting.

Preferred embodiments according to the present invention are shown in the following Figures and in a detailed specification, but are not intended to limit the present invention thereto.

In the Figures:

FIG. 1 shows a preferred diagnostic system for determining a clinically relevant severity of a sleep-related breathing disorder of a patient, emitting breathing noises which are recorded by a sensor unit and transformed into a patient signal, having a first detector unit detecting respiratory events in the patient signal and generating a respective respiratory event signal therefrom and a second detector unit which determines the clinically relevant severity of the sleep-related breathing disorder from the respiratory event signal;

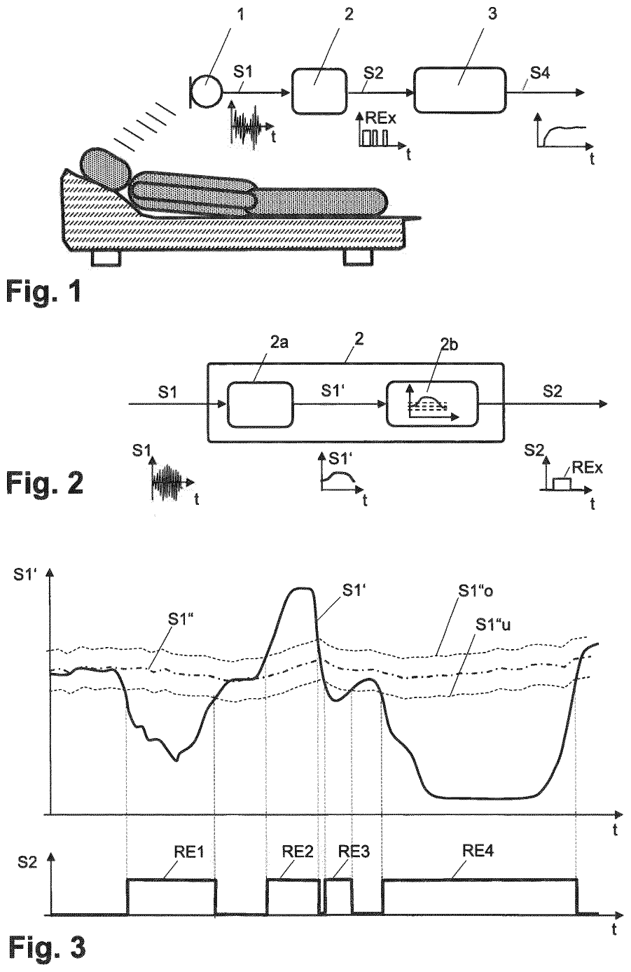

FIG. 2 shows a preferred embodiment of the first detector with first signal processing generating an intensity signal from the patient signal and second signal processing which recognizes the respiratory events in the intensity signal and outputs them as a respiratory event signal;

FIG. 3 shows, in the upper portion, a graph of an exemplary intensity signal over time with a chain-dotted prediction signal of the patient signal in case of non-disturbance and dashed upper and lower lines of a threshold value signal, which are generated from the prediction signal for threshold value detection of the respective respiratory events; and in the lower portion, a resulting respiratory event signal generated by the upper intensity signal with the lower and upper threshold value signals;

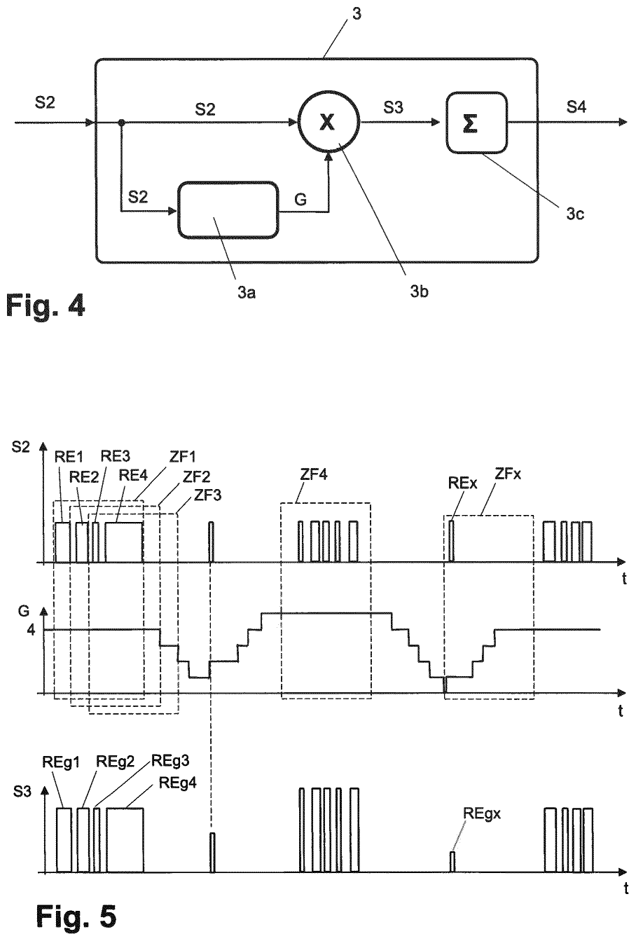

FIG. 4 shows a preferred embodiment of the second detector with a unit for weighting determination, which unit generates a weighting signal from the respiratory event signal, with a weighting unit which weights the respiratory event signal with the weighting signal and generates a weighted respiratory event signal therefrom, and with a severity detection unit which determines the clinically relevant severity of the sleep-related breathing disorder from the weighted respiratory event signal;

FIG. 5 shows an upper diagram with an exemplary respiratory event signal over time with different dashed-line time windows;
a centre diagram with a weighting signal generated by a preferred weighting detection which is proportional to a maximum number of respiratory events which can be recorded in a respective time window; and
a lower diagram showing the weighted respiratory event signal as it preferably would be generated by the weighting unit;

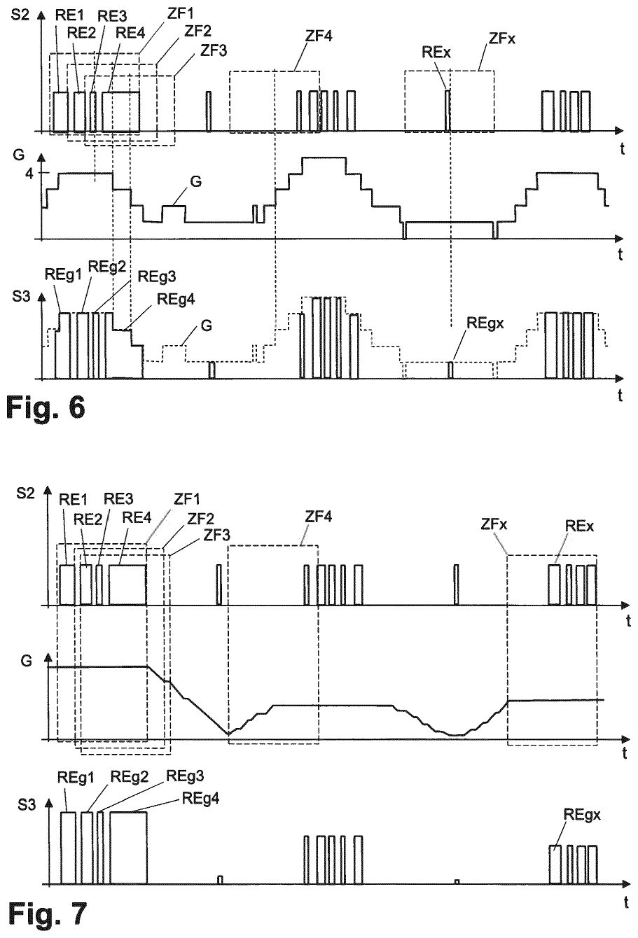

FIG. 6 shows an upper diagram with the exemplary respiratory event signal over time with different dashed-line time windows sliding over time;
a centre diagram with a different weighting signal generated by a different preferred weighting detection which is determined from the respiratory event signal proportionally to a number of respiratory events within a corresponding time window; and
a lower diagram showing the weighted respiratory event signal as it would preferably be generated by the weighting unit;

FIG. 7 shows an upper diagram with the exemplary respiratory event signal over time with different dashed-line time windows;
a centre diagram with an additional weighting signal generated by further preferred weighting detection and proportional to an energy of the respiratory event signals which can be captured by a respective time window; and
a lower diagram showing the weighted respiratory event signal as it would preferably be generated by the additional weighting unit.

### DETAILED DESCRIPTION OF EMBODIMENTS

FIG. 1 schematically shows a preferred diagnostic system for determining a clinically relevant severity S4 of a sleep-related breathing disorder from a patient signal of a sleeping patient. The diagnostic system comprises:
a) a sensor unit 1 which records at least one patient parameter and generates a corresponding patient signal S1 therefrom;
b) a first detector unit 2 with first signal processing 2a and second signal processing 2b, as shown by way of example in FIG. 2. The signal processing 2a generates an intensity signal S1′ from the patient signal S1, which can be, for instance, an RMS output or an averaged rectified signal. The second signal processing 2b is adapted to detect in the intensity signal S1′ respiratory events, Rex, and to generate therefrom a corresponding respiratory event signal S2; and
c) a second detector unit 3 adapted to determine from the respiratory event signal S2 the clinically relevant severity S4;
d) the second detector unit 3 according to the invention comprising
d1) a weighting determination unit 3a adapted to detect in the respiratory event signal S2 the respiratory events Rex and to determine for each respiratory event RE a corresponding weighting signal G by determining, for each respiratory event Rex within a corresponding predetermined time window ZFx which comprises the respective respiratory event Rex, respiratory events Rex close in time, with the respective weighting signal G being generated as a function depending on a number and/or an energy of the respective respiratory events Rex that are close in time, and at least partially rising;
d2) a weighting unit 3b which weights the respiratory event signal S2 with the weighting signal G and generates therefrom a weighted respiratory event signal S3 with accordingly weighted respiratory events; and
d3) a severity determining unit 3c which determines the clinically relevant severity S4 as an integral value over the weighted respiratory event signal S3 and preferably outputs it. Preferably, the integral value is determined using an integral of the weighted respiratory event signal S3 over a measurement time, divided by the measurement time in hours.

Preferably, the sensor unit 1 is a microphone, a microphone array and/or a different sensor unit 1 as is known from the state of the art. The patient signal S1 can comprise one or more signals. The at least one microphone is preferably an airborne-sound microphone to be able to record breathing and possibly snoring noises produced by the patient and convert them at least partially into the patient signal.

As schematically shown in FIG. 2, the first signal processing 2a generates from the patient signal S1 the intensity signal S1′ as an RMS output or a lowpass-filtered rectified patient signal. First signal processing 2a can also contain non-linear components for processing the patient signal S1, as is known from the state of the art. Preferably, first signal processing 2a averages the intensity signal S1′ over a predetermined time constant, such as 100-200 ms or 200-400 ms. Further preferably, the first signal processing 2a is adapted to connect adjacent local maximum values within the averaged intensity signal by a straight line. Even more preferably, the first signal processing 2a is adapted to maintain the intensity signal S1′ at a current hold value until a predefined release hold time has elapsed if the intensity signal determined over the time constant would otherwise be smaller than the hold value; if the intensity signal determined using the time constant exceeds the hold value, the output intensity signal S1′ would again correspond to the intensity signal determined using the time constant. The release hold time is set to be, for instance, 0.2-0.4 s or 0.4-1 s or 1-3 s.

The second signal processing 2b detects the respiratory events Rex for instance by means of a threshold value detector with a lower threshold-value signal S1″u which is adapted to detect whether the intensity signal S1′ falls below the lower threshold value signal, the corresponding respiratory event signal S2 being generated as shown by way of example in FIG. 3. The lower threshold value signal S1″u is preferably derived from a prediction signal S1″ of the intensity signal S1′. The prediction signal S1″ is generated such as to represent an undisturbed course of the intensity signal S1′, as is known from signal predictors known in the state of the art. One possible predictor for instance, in simple terms, is averaging, such as median-value generation, of the intensity signal S1′ over a previously elapsed time period. Preferably, when the prediction signal S1″ is determined, outliers of the intensity signal S1′ exceeding a predetermined variance are not taken into account. By an “undisturbed course” of the intensity signal S1′, it is intended that the intensity signal S1′ fluctuates within the variance fluctuation range around an average value, with no larger or smaller intensity signal values which would probably be correlated with a breathing disorder. The lower threshold value signal S1″u can be generated, for instance, from the prediction signal S1″, multiplied by a predefined factor smaller than 1. The lower threshold value signal S1″u can also be determined using a minimum value of the intensity signal S1′ which is determined by means of a previous predefined time period. This time period can be, for instance, 1-7 minutes. Predictor algorithms, adaptive filters, neural networks and the like are sufficiently known in the state of the art for determining the prediction signal S1″ as well as the lower S1″u and the upper threshold value signals S1″o and can be employed here.

It is conceivable to evaluate, by means of the threshold value detector, an upper threshold value signal S1″o by detecting whether the intensity signal S1′ exceeds this same threshold value signal, which could also generate a respiratory event signal. The upper threshold value signal S1″o can be generated, for instance, from the prediction signal S1″ multiplied by a predefined factor larger than 1. FIG. 3 shows an intensity signal S1′ by way of example, with a dash-dot line prediction signal S1″ and a lower S1″u and an upper threshold value signal S1″o. The respiratory event signal S2 shown below that becomes positive if it falls below the lower threshold value signal S1″u and if the upper threshold value signal S1″o is exceeded, with a first RE1, a second RE2, a third RE3 and a fourth respiratory event RE4 being mapped in the respiratory event signal S2.

In addition, second signal processing can be adapted to suppress respiratory events shorter than a predetermined minimum time or not to generate them at all so that they are not represented in the resultant respiratory event signal. Preferably, this minimum time is 10 seconds. For purposes of clarity, the wording “mapped” also means “output as a signal component”. Other first detector units 2 from the state of the art for generating the respiratory event signal S2 are also conceivable.

Preferably, the sensor unit 1 is part of a mobile radio unit which can wirelessly forward the patient signal S1 to the first detector unit 2. It is also conceivable that part of the first detector unit 2 and/or part of the second detector unit 3 are part of the mobile radio unit. Preferably, the second detector unit 3 is at least partly implemented on a system supported by a stationary microcontroller, where the clinically relevant severity S4 can preferably be sent back to the mobile radio unit as an evaluation signal.

FIG. 4 shows a preferred embodiment of the second detector 3, with a weighting determination unit 3a which generates a weighting signal G from the respiratory event signal S2. A weighting unit 3b then generates a weighted respiratory event signal S3 in dependence on the respiratory event signal S2 and the weighting signal G. In this process, the weighting unit 3b which is preferably embodied as a multiplication unit generates the weighted respiratory event signal S3 preferably from the respiratory event signal S2, multiplied by the weighting signal G. A severity determining unit preferably evaluates the weighted respiratory event signal S3 such that it sums up the respiratory event signal S3 and divides it by a measurement time over the summed-up respiratory event signal S3. The measurement time is preferably indicated in hours, with the clinically relevant severity of the sleep-related breathing disorder being determined as the number of respiratory events per hour. The clinically relevant severity is preferably repetitively calculated over a progressing measurement time. It is also possible to determine, and preferably indicate, the clinically relevant severity over one or a plurality of measurement times which are part of a total measurement time.

FIG. 5 shows determining of the weighted respiratory event signal S3 wherein the weighting determination unit 3a is preferably adapted so as to shift and arrange the respective predetermined time window ZFx in relation to the respective respiratory event REx on the time axis such that the largest possible number of respiratory events REx close in time lie completely or at least partially within the time window ZFx. For the first respiratory event RE1, for instance, a first time window ZF1 is arranged such that as to capture the first RE1, the second RE2, the third RE3 and the fourth respiratory event RE4, resulting in a number of 4 which are mapped in the correspondingly generated weighting signal G. Till a second time window ZF2, the four respiratory events RE1-RE4 can still be captured by a respective time window ZFx so that the weighting function till the time of an end of the second time window ZF2 is 4. From the second to a third time window ZF3, only the second RE2 up to the fourth respiratory event RE4 lie within a respective time window; therefore, the number and the weighting value G cover this time span 3. With a fourth time window ZF4, five respiratory events can be captured; the number and the weighting value are 5. At the time with the respiratory event REx, no respiratory event close in time can be captured; therefore, the weighting signal G at this time is 1. The resulting weighted respiratory event signal S3 is displayed on the bottom of the Figure, with a resulting weighted first REg1−fourth weighted REg4 having a value of 4 corresponding to the respective weighting signal G. It can be seen that in the summation over the respiratory events, singular respiratory events contribute less to the clinically relevant severity. As has been stated above, the correction or calibration factor can also be included in the weighting signal G.

Alternatively, the weighting determination unit 3a can be adapted to shift and arrange the respective time window ZFx for the corresponding respiratory event REx on the time axis in such a way that the largest possible energy of the respective respiratory event REx and of the respiratory events REx close in time are captured in the time window ZFx.

FIG. 6 shows another way of determining the weighted respiratory event signal S3 wherein the weighting determination unit 3a is preferably adapted to shift the predefined time window ZFx as a sliding time window along a time axis t of the respiratory event signal S2, each time determining the number of all respiratory events REx contained entirely or at least partly in the time window ZFx and mapping the number in the weighting signal G at a predefined time within the time window ZFx. The respective time window ZFx has a fixed relation to a point in time on the time axis t of the weighting signal G, with the time window being for instance one or one half time window length before the respective point in time on the time axis t of the weighting signal G. In contrast, with the method according to FIG. 5, the time window ZFx is shifted back and forth as long as the point in time still lies within the time window ZFx, where in each time window around the point in time on the time axis t of the weighting signal G the number and/or the energy of the respiratory event signal are determined.

In the example of FIG. 6, the time window lies centrally around the respective point in time and is also arranged shifted by half the time window length around the point in time. The first time window ZF1 contains at least partly the first RE1, the second RE2, the third RE3 and the fourth respiratory event RE4, the number of respiratory events being 4 and the respective weighting signal G accordingly being determined with the value of 4. The weighting signal G is determined accordingly as a sum of the respective number of respiratory events REx in the respective time window ZFx. Below, the resultant weighted respiratory event signal S3 is shown, with the first weighted respiratory event signal REg1 being partially weighted with a value of 3 and partially with a value of 4; the fourth respiratory event RE4 is generated, in accordance with the weighting signal G, as a weighted respiratory event signal REg4 partly with the value of 4, partly with the value of 3 and partly with the value of 2. The forming of an integral in determining the clinically relevant severity takes such weighted respiratory event signals S3, which change over a time, into account.

FIG. 7 shows a preferred way of determining the weighted respiratory event signal S3 wherein the weighting determination unit 3a is adapted to shift the predefined time window ZFx as a sliding time window along the time axis t of the respiratory event signal S2, each time determining an integral value over the energy of all respiratory events REx contained at least partly therein and mapping the integral value in the weighting signal G at the predefined time within the time window ZFx. Preferably, the predefined time within the time window ZFx is at the beginning or in the middle or at the end of the time window. In other words, the time window ZFx is arranged by half one time window length to the left or to the right or centrally to the predefined time. It is also conceivable that during the respective time window ZFx, both the energy and the number of respiratory events in the time window are evaluated and the weighting signal is accordingly determined in dependence on the energy and the number of respiratory events.

Preferably, the weighting determination unit 3a is adapted to determine the weighting signal G by such a function that the weighting signal G increases with the number and/or the energy of the corresponding respiratory event REx within the predetermined time window ZFx.

Preferably, the severity determining unit 3c determines the clinically relevant severity S4 as an integral value over the weighted respiratory event signal S3 in combination with a predefined correction factor. Preferably, the correction factor is predetermined from a plurality of respective correction factors as an average value by equating a plurality of integral values taken from training data sets from the weighted respiratory event signals S3, multiplied by the respective correction factors, to the respective actual clinically relevant severities S4 which have been determined by a person skilled in the art from the respiratory event signals S2 with the respective respiratory events REx. The correction factors can be determined by formation of quotients, in which way a correction factor which has been averaged or determined otherwise can be set which is implemented in the diagnostic system, for instance in the severity determining unit 3c.

Preferably, the diagnostic system also has an output unit, for example a monitor displaying the clinically relevant severity.

For purposes of clarity, the weighting signal G is synchronous in time with the respiratory event signal S2. The respiratory event REx to which the respective time window refers lies always at least partially within the time window. Preferably, the time window begins half a time window length before the middle of the time window and ends half a time window length after the middle of the time window. The time window has a predefined length of preferably 1-5 min. or 5-10 min.

The weighting signal G, the patient signal S1, the intensity signal S1′, the prediction signal of the patient signal S1″, the lower threshold value signal S1″u, the upper threshold value signal S1″o, the respiratory event signal S2 and the weighted respiratory event signal S3 are all preferably variable signals dependent on time t. The clinically relevant severity S4 can be determined and output at an end of the measurement time as a value; however, it is also conceivable that the clinically relevant severity over time t is determined in portions and is therefore also time-dependent.

Weighting of the respiratory event signal S2 with the weighting signal G can be understood to be a multiplication or another linear or non-linear function of the respiratory event signal S2 and the weighting signal G. Weighting functions known in the state of the art, preferably linear functions or square functions, can be employed.

### Under a Recognition a Detecting Is Meant.

For purposes of clarity, the indications “top” and “bottom” are understood to be relative locations in the vertical direction, as shown in the Figures.

For clarity purposes, it is also pointed out that indefinite articles in combination with an object or numerals, for example “one”, are not intended to limit the object to exactly one, but to indicate “at least one”. This applies for all indefinite articles as “one” etc.

It is understood that when an element is indicated as being located “on” another element, “connected”, “coupled” or “in contact” therewith, the element can be either positioned directly on the other element, connected or coupled therewith or intermediate elements can be present as well which are either simply located therebetween or connect or couple the element to the other element or keep them in contact. If, on the other hand, an element is indicated as being positioned “directly on” another element, “directly connected”, “directly coupled” or “directly in contact” therewith, it is understood that no intermediate elements are present. Similarly, if a first element is indicated as being “in electrical contact with a second element” or “electrically coupled” therewith, there is an electrical path which allows the flow of current between the first and the second element. The electrical path can include capacitors, coupled inductivities and/or other elements which allow the flow of current between the conductive elements even without direct contact.

Although the expressions “first”, “second” etc. can be used herein to designate different elements, components, areas and/or portions, these elements, components, areas and/or portions are not limited by these expressions. The expressions are rather used to distinguish a component, element, area or portion from another. Consequently, a first element, component, area or portion discussed below can be called a second element, component, area or portion without deviating from the teachings of the present invention.

Embodiments of the invention are described herein with reference to cross-sectional views which are schematic representations of embodiments of the invention. Therefore, the actual thickness of the components may vary and deviations from the forms in the Figures, for example due to manufacturing methods and/or tolerances, are to be expected. Embodiments of the invention are not to be understood as being limited to the specific forms of the areas shown herein but are to include deviations of the forms resulting, for example, from the type of production. A region shown as a square or rectangular region typically also has rounded or curved features due to normal manufacturing tolerances. Therefore, the regions shown in the Figures are schematic and their forms are not intended to represent the exact shape of a region of a device or to limit the scope of protection of the invention.

Relational expressions, such as “inner”, “outer”, “upper”, “above”, “over”, “below” and similar expressions can be used to indicate a relation between a layer or different region and another layer or region. It is understood that these expressions are to comprise different orientations of the device in addition to the one shown in the Figures.

Concerning the expression “comprise”, it is stated for purposes of clarity that when a first device component is said to comprise a second device component, this means that the first device component “contains” the second device component and not necessarily that the first component encloses the second one in terms of arrangement, unless an arrangement in term of position and form is described; the same applies to a method which can comprise one or more method steps.

Other possible embodiments are described in the following Claims. In particular, the various features of the embodiments described above can be combined unless they are technically mutually exclusive.

The reference numbers indicated in the Claims are only given for more clarity and do not limit the Claims to the embodiments shown in the Figures in any way.

### List of Reference Numbers

1 sensor unit, for example microphone unit
2 first detector unit
2a first signal processing, preferably RMS filter
2b second signal processing, preferably respiratory signal detector
3 second detector unit
3a weighting determination unit
3b weighting unit
3c severity determining unit
G weighting signal from weighting values
REx, RE1-RE4 respiratory event, first-fourth respiratory event
REgx, REg1-REg4 weighted respiratory event, first-fifth weighted respiratory event
S1 patient signal, for example microphone signal
S1′ intensity signal
S1″ prediction signal of the patient signal
S1″u lower threshold value signal
S1″o upper threshold value signal
S2 respiratory event signal
S3 weighted respiratory event signal
S4 clinically relevant severity
t time axis
ZFx, ZF1-ZF4 time window, first-fourth time window

1. A Diagnostic system for determining a clinically relevant severity of a sleep-related breathing disturbance from a patient signal of a sleeping patient, comprising
a) a sensor unit being adapted to records at least one patient parameter and to generates a corresponding patient signal therefrom;
b) a first detector unit having a first signal processing and being adapted to generates an intensity signal from the patient signal and having a second signal processing being adapted to detects respiratory events in the intensity signal and to generates a corresponding respiratory event signal; and
c) a second detector unit adapted to determine the clinically relevant severity from the respiratory event signal;
characterized in that
d) the second detector unit comprises:
d1) a weighting determination unit adapted to detect the respiratory events in the respiratory event signal and to determine, for each respiratory event, a corresponding weighting signal by determining for the respective respiratory event close respiratory events lying within a corresponding predetermined time window which also comprises the respective respiratory event, and generating the respective weighting signal depending on a number and/or an energy of the respective respiratory events that are close in time, and at least partially rising;
d2) a weighting unit being adapted to weights the respiratory event signal with the weighting signal and to generates a weighted respiratory event signal with accordingly weighted respiratory events therefrom; and
d3) a severity determining unit being adapted to determines the clinically relevant severity as an integral value over the weighted respiratory event signal.

2. The Diagnostic system according to claim 1, the weighting determination unit being adapted to shift and arrange the respective predetermined time window for the respective respiratory event on the time axis in such a way that a largest possible number of respiratory events close in time at least partially lies within the time window.

3. The Diagnostic system according to claim 1, the weighting determination unit being adapted to shift and arrange the respective predetermined time window for the respective respiratory event on the time axis in such a way that a highest possible energy of the respective respiratory event and of the respiratory events close in time lies within the time window.

4. The Diagnostic system according to claim 1, the weighting determination unit being adapted to shift the predetermined time window as a sliding time window along a time axis of the respiratory event signal, each time determining the number of all respiratory events at least partially contained within the time window and mapping the number in the weighting signal at a predefined time within the time window.

5. The Diagnostic system according to claim 1, with the weighting determination unit being adapted to shift the predetermined time window as a sliding time window along the time axis of the respiratory event signal, each time forming an integral value over the energy of all respiratory events at least partially contained therein and mapping the integral value in the weighting signal at the predefined time within the time window.

6. The Diagnostic system according to claim 1, with the weighting determination unit being adapted to determine the weighting signal in dependence on the number and/or the energy of the respective respiratory event and of the respiratory events close in time within the predetermined time window such that the weighting signal increases with the number and/or the energy.

7. The Diagnostic system according to claim 1, with the severity determining unit being adapted to determine the clinically relevant severity as an integral value over the weighted respiratory event signal in combination with a predefined correction factor.

8. The Diagnostic system according to claim 7, wherein the severity determining unit is adapted to determine the correction factor from a plurality of respective correction factors as an average value by equating a plurality of integral values taken from training data sets from the weighted respiratory event signals, with the respective correction factors, to the respective actual clinically relevant severities which have been determined by a person skilled in the art from the respiratory event signals with the respective respiratory events, wherein the correction factors can be determined by formation of quotients.

9. The Diagnostic system according to claim 1, the weighting determination unit being adapted to generate the weighting signal depending on the number and/or the energy of the respective respiratory events that are close in time within a major range of values of the number and/or the energy of the respective respiratory events close in time, increasing therewith.

10. The Diagnostic system according to claim 4, with the weighting determination unit being adapted to shift the predetermined time window as a sliding time window along the time axis of the respiratory event signal, each time forming an integral value over the energy of all respiratory events at least partially contained therein and mapping the integral value in the weighting signal at the predefined time within the time window.

11. The Diagnostic system according to claim 4, with the severity determining unit being adapted to determine the clinically relevant severity as an integral value over the weighted respiratory event signal in combination with a predefined correction factor.
